(19) United States
(12) Patent Application Publication
(10) Pub. No.: US 2003/0215431 A1
(43) Pub. Date: Nov. 20, 2003

(54) Management of mucosal viscosity by TFF monomer peptides

(76) Inventors: Lars Thim, Gentofte (DK); Steen Seier Poulsen, Allerod (DK)

Correspondence Address: Reza Green, Esq., Novo Nordisk Pharmaceuticals, Inc., 100 College Road West, Princeton, NJ, 08540, US

(21) Appl. No.: 10/364,751

(22) Filed: Feb. 11, 2003

Related U.S. Application Data
(60) Provisional application No. 60/361,374, filed on Feb. 26, 2002.

(30) Foreign Application Priority Data
Feb. 11, 2002 (DK) 2002 00200

(51) Int. Cl. A61K 38/46

(52) U.S. Cl. 424/94.6

(57) ABSTRACT

Mammalian trefoil factors (TFFs) constitute a group of three peptides (TFF1, TFF2 and TFF3) widely distributed in the gastrointestinal tract. These peptides are characterized as containing one (TFF1 and TFF3) or two (TFF2) trefoil domains. The present invention relates to the use of human TFF1 monomer and TFF3 monomer peptides for improving rheological properties of mucin solutions. TFF monomer peptides have been found to decrease the viscosity and elasticity of different mucin solutions when the TFF monomer peptides are in competition with TFF dimer peptides, e.g. TFF2. Trefoil factor 1 (TFF1) and trefoil factor 3 (TFF3) monomers and pharmaceutical compositions comprising TFF monomers are useful for decreasing the viscosity of mucin in mucus layers, and the repair of damaged mucus layers in the gastrointestinal tract (mouth, oesophagus, stomach, small and large intestine, colon) the respiratory passages, the eye, the urinary system (including the bladder) and the cervis uteri.

### CROSS-REFERENCE TO RELATED APPLICATIONS

This application claims priority under 35 U.S.C. 119 of Danish application no. PA 2002 00200 filed Feb. 11, 2002 and U.S. provisional application no. 60/361,374 filed Feb. 26, 2002, the contents of which are fully incorporated herein by reference.

### FIELD OF THE INVENTION

The present invention relates to the use of trefoil factor 1 (TFF1) and trefoil factor 3 (TFF3) monomers and a pharmaceutical composition comprising TFF monomers for decreasing the viscosity of mucin in mucus layers and the repair of damaged mucus layers in the gastrointestinal tract (mouth, oesophagus, stomach, small and large intestine, colon) the respiratory passages, the eye, the urinary system (including the bladder) and the cervis uteri.

### BACKGROUND OF THE INVENTION

Mammalian trefoil factors (TFFs) constitute a group of three peptides (TFF1, TFF2 and TFF3) widely distributed in the gastrointestinal tract. These peptides are characterised by containing one (TFF1 and TFF3) or two (TFF2) trefoil domains. A trefoil domain is defined as a sequence of 38 or 39 amino acid residues in which six cysteines are disulphide-linked in a 1-5, 2-4 and 3-6 configuration. The trefoil peptides are expressed in the gastrointestinal tract in a tissue specific manner. In humans TFF1 and TFF2 are expressed in mucus producing cells in the stomach and duodenum, whereas TFF3 is primarily expressed in goblet cells in the small and large intestine. In the case of gastric ulceration or inflammatory bowel disease the expression of trefoil peptides is highly upregulated. This suggest that trefoil peptides may have a repair function for damages in the gastrointestinal tract thus acting as naturally occurring healing factors. The importance of TFFs for normal mucosal function have also been investigated by two gene knock-out studies in which the genes encoding TFF1 and TFF3, respectively, were deleted by gene-targeting techniques. The TFF3 knock-out mice had impaired mucosal healing and died from extensive colitis after oral administration of dextran sulphate a situation that could be circumvented by luminal administration of recombinant TFF3. Although several studies have documented a protection or healing effect of trefoil peptides in gastric ulceration and colitis models the detailed mechanism of action is still largely unknown. One of the theories is that trefoil peptides together with mucins form stable gel complexes resistant to mechanical stress and gastrointestinal proteases. Although no direct evidence for such gel formation has so far been given some studies have indicated an interaction/binding between trefoil peptides and mucins.

The cloning of rat and human single-domain TFF3 (ITF) and the use of this peptide in the treatment of gastrointestinal injury is described in WO 92/14837.

### DESCRIPTION OF THE INVENTION

The present invention relates to the use of human TFF1 monomer and TFF3 monomer peptides for improving rheological properties of mucin solutions. TFF monomer peptides have by the present inventors been found to decrease the viscosity and elasticity of different mucins solutions, when the TFF monomer peptides are in competition with TFF dimer peptides, e.g. TFF2. The viscosity and elasticity of mucin in mucus layers are correlated to physiological and pathophysiological conditions.

The present invention discloses the mechanism by which the TFF monomer peptides exert their biological activity, which are documented by a direct effect of TFF monomer peptides on the viscosity and elasticity of mucin solutions. The TFF monomer peptides significantly decrease the viscosity of mucin solutions, when the TFF monomer peptides are in competition with TFF dimer peptides, e.g. TFF2. The net effect is a decrease in the viscosity of several times and can be visualised when a viscous gel-like substance is converted to a more liquid mucin solution.

The TFF monomer peptides have by the present inventors been found to be usefull for decreasing the viscosity and elasticity of mucus layers, which can be used in the treatment of many different indications, where abnormalities in existing mucus layers are present. The advantage over known therapies is, that treatment with TFF monomer peptides represents a specific treatment at the site of injury without major side effects. It has to be understood, that TFF monomer and dimer peptides most probably in vivo are in constant equilibrium in order to have the most optimal viscosity and elasticity for a given mucus layer. To regulate or spefically decrease the viscosity the TFF monomer peptides may be given alone or in combination with other viscosity regulating substances.

For local and luminal applications TFF monomer peptides can decrease the viscosity and elastic properties of mucin in mucus layers, which may be usefull in many different indications:

1) For the treatment of diseases in the gastrointestinal tract: Mucositis in the oral mucosa caused by e.g. irradiation involving the salivary glands or dry mouth (xerostomia) in Sjögrens syndrome or induced by drugs.

2) Treatment of diseases in the small and large intestine causing mucosal ulcerations to make the mucus layer more confluent in order to coat the ulcerated surfaces.—Ulcerative colitis, Crohn's disease, pseudomembranous colitis. Obstipation to decrease the viscosity of the colonic secretions, irritable bowel syndrome, and cystic fibrosis.

3) For the treatment of diseases in the respiratory system: Local application in the nose against viscous mucus secretions in sinusitis and common cold causing nasal obstruction. In inhalation form as mucolytic in diseases of the lungs causing viscous secretions and sputum, asthma, acute and chronic bronchitis, alpha-1 antitrypsin deficiency and cystic fibrosis.

4) For the treatment of diseases in the urogenital system: TFF monomers are present in the glands of the uterine cervix. Too viscous secretions might be a cause of decreased fertility and might be treated with TFF monomers. At artificial insemination the follicle fluid might be treated with TFF monomers to facilitate the penetration of the semen.

5) For the protection of the stomach against acute stress induced gastric ulcers secondary to trauma, shock, large operations, renal or lever diseases, or gastritis caused by treatment with aspirin or other NSAIDS, steroids or by alcohol.

6) For the protection of the small intestinal and colonic mucosa in Crohns disease and ulcerative colitis.

7) In eye droplets to decrease the viscosity of lacrimal fluid in patients with keratoconjunctivitis sicca/Sjögren's syndrome or “dry eyes” for other reasons.

8) Local application especially in the knee joints to decrease the viscosity of the synovial fluid in osteoarthritis and following joint replacement.

TFF monomer peptides may also be used for parenteral applications:

Parenteral TFF monomer is taken up by cells associated with stem cells in the gastrointestinal tract. It can be used for protection of the stomach against stress-induced damage and the stomach and intestine against damage following irradiation or chemotherapy or in the treatment of acute excerbations in ulcerative colitis or Crohn's disease. Injected TFF monomer peptide is excreted intact in urine and may increase the defence mechanism of the urinary bladder by binding to the layer of mucopolysaccharids that coat the urothelium and thereby interfere with the adherence of bacteria in chronic bladder infections, in patients with catheter or interstitial cystitis, or interfere with the binding of urinary growth factors in papillomas or cancer of the bladder.

In a first aspect, the present invention relates to a pharmaceutical composition for decreasing the viscosity of mucus layers in mammals, the composition comprising a TFF monomer peptide or a pharmaceutically acceptable salt thereof.

By “TFF monomer peptides” or “a TFF monomer peptide” is meant a protein that is substantially homologous to human TFF1 or human TFF3 in monomer forms. FIG. 1 shows TFF1 and TFF3 in the monomer form. The term TFF monomer peptides also includes derivatives and analogs of naturally occurring TFF monomer peptides. Analogs can differ from naturally occurring TFF monomer by amino acid sequence differences or by modifications that do not affect sequence, or by both. Analogs of the invention will generally exhibit at least 70%, more preferably 80%, more preferably 90%, and most preferably 95% or even 99%, sequence identity with a naturally occurring TFF monomer sequence.

The terms “decreasing the viscosity” or decrease in viscosity” as used herein means any reduction in viscosity η (Pa s). This may be measured as described in example 1.

In one embodiment the viscosity is decreased by more than 3%. In one embodiment the viscosity is decreased by more than 5%. In one embodiment the viscosity is decreased by more than 10%. In one embodiment the viscosity is decreased by more than 15%. In one embodiment the viscosity is decreased by more than 20%. In one embodiment the viscosity is decreased by more than 30%. In one embodiment the viscosity is decreased by more than 40%. In one embodiment the viscosity is decreased by more than 50%. In one embodiment the viscosity is decreased by more than 60%. In one embodiment the viscosity is decreased by more than 70%. In one embodiment the viscosity is decreased by more than 80%. In one embodiment the viscosity is decreased by more than 90%. In one embodiment the viscosity is decreased by more than 100%. In one embodiment the viscosity is decreased by more than 150%.

Modifications include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps, e.g., by exposing the polypeptide to enzymes that affect glycosylation derived from cells that normally provide such processing, e.g., mammalian glycosylation enzymes. Also embraced are versions of the same primary amino acid sequence that have phosphorylated amino acid residues, e.g., phosphotyrosine, phosphoserine, or phosphothreonine.

In addition to substantially full-length polypeptides, the term TFF monomer peptide, as used herein, includes biologically active fragments of the polypeptides. As used herein, the term “fragment,” as applied to a polypeptide, will ordinarily be at least 10 contiguous amino acids, typically at least 20 contiguous amino acids, more typically at least 30 contiguous amino acids, usually at least 40 contiguous amino acids, preferably at least 50 contiguous amino acids, and most preferably at least 60 to 80 or more contiguous amino acids in length. The ability of a candidate fragment to exhibit a biological activity of a TFF monomer peptide can be assessed by methods known to those skilled in the art. Also included in the term “fragment” are biologically active TFF monomer peptides containing amino acids that are normally removed during protein processing, including additional amino acids that are not required for the biological activity of the polypeptide, or including additional amino acids that result from alternative mRNA splicing or alternative protein processing events.

A TFF monomer peptide, including a fragment, or analog is biologically active if it exhibits a biological activity of a naturally occurring TFF monomer, e.g., the ability to alter viscosity or elasticity of mucin in mucus layers in a mammal.

The term “glycosylation”, as used herein, means the post-translational modification of a peptide, wherein a carbohydrate molecule is covalently attached to the peptide. The glycosylation may take place in a eucaryotic host cell, such as a yeast cell or it may be done by chemical linkage in vitro after production of the peptide in a cell, e.g. the peptide could be produced in a bacteria and glycosylated in vitro afterwards.

In a second aspect, the present invention relates to the use of a TFF monomer peptide for the preparation of a medicament for decreasing the viscosity of mucus layers in mammals.

In a third aspect, the present invention relates to a method for in vivo decrease in viscosity of mucus layers in a subject, the method comprising administering to the subject a composition comprising

a) a pharmaceutically acceptable carrier or diluent,

b) a therapeutically effective amount of a TFF monomer peptide, and optionally

c) a mucin glycoprotein preparation.

The term “in vivo” as used herein refers to any application on or inside a living organism.

In another aspect, the present invention relates to the use of a TFF monomer peptide for the treatment of conditions characterized by increased viscosity of mucus layers in mammals.

The term “treatment”, as used herein, means the administration of an effective amount of a therapeutically active compound of the invention with the purpose of preventing any symptoms or disease state to develop or with the purpose of curing or easing such symptoms or disease states already developed. The term “treatment” is thus meant to include prophylactic and protective treatment. The symptoms or disease state includes but is not limited to diseases, e.g. gastric ulcers or asthma, inherited biological disorders or conditions induced by damaging by external stimuli, e.g. Inhalation of toxic or acidic chemical.

In one embodiment, the present invention relates to a method for an ex vivo decrease in viscosity of a mucus layer, the method comprising administering to a mucus layer a composition comprising

a) a therapeutically effective amount of a TFF monomer peptide, and optionally

b) a mucin glycoprotein preparation.

The term “ex vivo” as used herein refers to any application outside the living body of a mammal.

In one embodiment, the present invention relates to a method for an in vitro decrease in viscosity of a solution comprising mucin, the method comprising administering to said solution a composition comprising

a) an effective amount of a TFF monomer peptide, and optionally

c) a mucin glycoprotein preparation.

In one embodiment of the invention, the mammal is human.

Another embodiment of the present invention relates to a pharmaceutical composition for local application.

In a further embodiment the present invention relates to a pharmaceutical composition for luminal application.

In a further embodiment the present invention relates to a pharmaceutical composition for parenteral administration.

In a further embodiment the present invention relates to a pharmaceutical composition for oral administration.

In a further embodiment the present invention relates to a pharmaceutical composition further comprising a mucin glycoprotein preparation.

In a further embodiment the present invention relates to a pharmaceutical composition for the treatment of oral mucosa.

In a further embodiment the present invention relates to a pharmaceutical composition for the treatment of patients with reduced secretion of saliva. In one embodiment, the reduced secretion of saliva is mucositis. In one embodiment, the reduced secretion of saliva is caused by irradiation therapy, treatment with drugs, e.g. anticholinergics or Sjögrens syndrome.

In a further embodiment, the present invention relates to a pharmaceutical composition for the treatment of patients receiving irradiation therapy.

In a further embodiment, the present invention relates to a pharmaceutical composition for the treatment of patients treated with anticholinergics.

In a further embodiment, the present invention relates to a pharmaceutical composition for the treatment of patients with Sjögrens syndrome.

In a further embodiment the present invention relates to a pharmaceutical composition for the treatment of the respiratory passages.

In a further embodiment the present invention relates to a pharmaceutical composition for decreasing the viscosity of secretions in sinusitis or common cold causing nasal obstruction.

In a further embodiment, the present invention relates to a pharmaceutical composition for the treatment of patients with common cold.

In a further embodiment, the present invention relates to a pharmaceutical composition for the treatment of patients with nasal obstruction.

In a further embodiment, the present invention relates to a pharmaceutical composition for the treatment of patients with allergic rhinitis.

In a further embodiment, the present invention relates to a pharmaceutical composition for the treatment of patients with diseases of the lungs causing viscous secretions and sputum, asthma, acute or chronic bronchitis, alpha-1 antitrypsin deficiency and cystic fibrosis.

In a further embodiment, the present invention relates to a pharmaceutical composition for the treatment of patients with viscous secretions in the lungs.

In a further embodiment, the present invention relates to a pharmaceutical composition for the treatment of patients with viscous sputum.

In a further embodiment, the present invention relates to a pharmaceutical composition for the treatment of patients with asthma.

In a further embodiment, the present invention relates to a pharmaceutical composition for the treatment of patients with acute or chronic bronchitis.

In a further embodiment, the present invention relates to a pharmaceutical composition for the treatment of patients with alpha-1 antitrypsin deficiency.

In a further embodiment, the present invention relates to a pharmaceutical composition for the treatment of patients with cystic fibrosis.

In a further embodiment the present invention relates to a pharmaceutical composition for the treatment of the respiratory tract.

In a further embodiment the present invention relates to a pharmaceutical composition for the treatment of the respiratory tract following accidental inhalation of irritants.

In a further embodiment the present invention relates to a pharmaceutical composition for the treatment of the respiratory tract following accidental inhalation of gases, dusts or fumes.

In a further embodiment the present invention relates to a pharmaceutical composition for the treatment of the gastrointestinal tract. In a further embodiment the present invention relates to a pharmaceutical composition for the treatment of oesophagus. In one embodiments the present invention relates to a pharmaceutical composition for the treatment of the distal part of the oesophagus.

In a further embodiment the present invention relates to a pharmaceutical composition for protection against acid secretions from the stomach.

In a further embodiment the present invention relates to a pharmaceutical composition for protection against acid secretions from the stomach in reflux oesophagi's.

In a further embodiment the present invention relates to a pharmaceutical composition for protection against acid secretions from the stomach in hiatus hernia.

In a further embodiment the present invention relates to a pharmaceutical composition for protection against acid secretions from the stomach in Barrets oesophagus.

In a further embodiment the present invention relates to a pharmaceutical composition for the treatment of the stomach.

In a further embodiment the present invention relates to a pharmaceutical composition for treatment of stress induced gastric ulcers. In one embodiment the stress induced gastric ulcers is secondary to trauma. In another embodiment the stress induced gastric ulcers is secondary to shock. In a further embodiment the stress induced gastric ulcers is secondary to large operations. In a further embodiment the stress induced gastric ulcers is secondary to renal diseases. In a further embodiment the stress induced gastric ulcers is secondary to lever diseases. In a further embodiment the stress induced gastric ulcers is secondary to treatment with aspirin, other non-steroidal anti-inflammatory drugs (NSAIDS), steroids or alcohol.

In a further embodiment the present invention relates to a pharmaceutical composition for the treatment of the small intestinal mucosa.

In a further embodiment the present invention relates to a pharmaceutical composition for the treatment of the colonic mucosa.

In a further embodiment the present invention relates to a pharmaceutical composition for the treatment of Crohns disease.

In a further embodiment the present invention relates to a pharmaceutical composition for the treatment of ulcerative colitis.

In a further embodiment the present invention relates to a pharmaceutical composition for the treatment of pseudomembranous colitis.

In a further embodiment the present invention relates to a pharmaceutical composition for the treatment of obstipation.

In a further embodiment the present invention relates to a pharmaceutical composition for the treatment of irritable bowel syndrome.

In a further embodiment the present invention relates to a pharmaceutical composition for the treatment of the eye.

In a further embodiment the present invention relates to a pharmaceutical composition for decreasing the viscosity of lacrimal fluid.

In a further embodiment the present invention relates to a pharmaceutical composition for decreasing the viscosity of lacrimal fluid in patients with keratoconjunctivitis sicca.

In a further embodiment the present invention relates to a pharmaceutical composition for decreasing the viscosity of lacrimal fluid in patients with Sjögren's syndrome.

In a further embodiment the present invention relates to a pharmaceutical composition for decreasing the viscosity of lacrimal fluid in patients with dry eyes.

The term “dry eyes”, as used herein, means any condition where the eyes feels dry.

In a further embodiment the present invention relates to a pharmaceutical composition in eye droplets.

In a further embodiment the present invention relates to a pharmaceutical composition for the treatment of a joint.

In a further embodiment the present invention relates to a pharmaceutical composition for the treatment of the knee joints.

In a further embodiment the present invention relates to a pharmaceutical composition for the treatment of a disease state in a joints, wherein the diseased state is increased viscosity of the joint.

In a further embodiment the present invention relates to a pharmaceutical composition for the treatment of a disease state in a joints, wherein the diseased state is increased viscosity of the synovial fluid in osteoarthritis or following joint replacement.

In a further embodiment the present invention relates to a pharmaceutical composition for decreasing the viscosity of the synovial fluid.

In a further embodiment the present invention relates to a pharmaceutical composition for decreasing the viscosity of the synovial fluid in osteoarthritis.

In a further embodiment the present invention relates to a pharmaceutical composition for decreasing the viscosity of the synovial fluid following joint replacement.

In a further embodiment the present invention relates to a pharmaceutical composition for the treatment of the bladder.

In a further embodiment the present invention relates to a pharmaceutical composition for the treatment of patients with catheter.

In a further embodiment the present invention relates to a pharmaceutical composition for the treatment of infections. In one embodiment the infection is a cronic infection of the bladder.

In a further embodiment the present invention relates to a pharmaceutical composition for the treatment of interstitial cystitis.

In a further embodiment the present invention relates to a pharmaceutical composition for the treatment of papillomas.

In a further embodiment the present invention relates to a pharmaceutical composition for the treatment of cancer.

In a further embodiment the present invention relates to a pharmaceutical composition for the treatment of the urogenital system.

In a further embodiment the present invention relates to a pharmaceutical composition for the treatment of the uterine cervix.

In a further embodiment the present invention relates to a pharmaceutical composition for the treatment of infertility.

In a further embodiment the present invention relates to a pharmaceutical composition to facilitate the penetration of the semen.

In a further embodiment of the invention, the TFF monomer peptide is recombinant human TFF1.

In a further embodiment of the invention, the TFF monomer peptide is recombinant human TFF3.

In a further embodiment of the invention, the TFF monomer peptide is glycosylated.

TFF monomer peptides are typically produced by recombinant DNA techniques. To this end, a DNA sequence encoding the TFF monomer peptide may be isolated by preparing a genomic or cDNA library and screening for DNA sequences coding for all or part of the peptide by hybridization using synthetic oligonucleotide probes in accordance with standard techniques (cf. Sambrook et al., Molecular Cloning: A Laboratory Manual, Cold Spring Harbor Laboratory, Cold Spring Harbor, N.Y., 1989). For the present purpose, the DNA sequence encoding the peptide is preferably of human origin, i.e. derived from a human genomic DNA or cDNA library.

The DNA sequences encoding the TFF monomer peptides may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. According to the phosphoamidite method, oligonucleotides are synthesized, e.g. in an automatic DNA synthesizer, purified, annealed, ligated and cloned in suitable vectors.

The DNA sequences may also be prepared by polymerase chain reaction using specific primers, for instance as described in U.S. Pat. No. 4,683,202, Saiki et al., Science 239 (1988), 487-491, or Sambrook et al., supra.

The DNA sequences encoding the TFF monomer peptides are usually inserted into a recombinant vector which may be any vector, which may conveniently be subjected to recombinant DNA procedures, and the choice of vector will often depend on the host cell into which it is to be introduced. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the TFF monomer peptide is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the polypeptide.

The promoter may be any DNA sequence, which shows transcriptional activity in the host cell of choice and may be derived from genes encoding proteins either homologous or heterologous to the host cell.

Examples of suitable promoters for directing the transcription of the DNA encoding the TFF monomer peptide in mammalian cells are the SV40 promoter (Subramani et al., Mol. Cell Biol. 1 (1981), 854-864), the MT-1 (metallothionein gene) promoter (Palmiter et al., Science 222 (1983), 809-814) or the adenovirus 2 major late promoter.

An example of a suitable promoter for use in insect cells is the polyhedrin promoter (U.S. Pat. No. 4,745,051; Vasuvedan et al., FEBS Lett. 311, (1992) 7-11), the P10 promoter (J. M. Vlak et al., J. Gen. Virology 69, 1988, pp. 765-776), the Autographa californica polyhedrosis virus basic protein promoter (EP 397 485), the baculovirus immediate early gene 1 promoter (U.S. Pat. No. 5,155,037; U.S. Pat. No. 5,162,222), or the baculovirus 39K delayed-early gene promoter (U.S. Pat. No. 5,155,037; U.S. Pat. No. 5,162,222).

Examples of suitable promoters for use in yeast host cells include promoters from yeast glycolytic genes (Hitzeman et al., J. Biol. Chem. 255 (1980), 12073-12080; Alber and Kawasaki, J. Mol. Appl. Gen. 1 (1982), 419-434) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4c (Russell et al., Nature 304 (1983), 652-654) promoters.

Examples of suitable promoters for use in filamentous fungus host cells are, for instance, the ADH3 promoter (McKnight et al., The EMBO J. 4 (1985), 2093-2099) or the tpiA promoter. Examples of other useful promoters are those derived from the gene encoding A. oryzae TAKA amylase, Rhizomucor miehei aspartic proteinase, A. niger neutral α-amylase, A. niger acid stable α-amylase, A. niger or A. awamori glucoamylase (gluA), Rhizomucor miehei lipase, A. oryzae alkaline protease, A. oryzae triose phosphate isomerase or A. nidulans acetamidase. Preferred are the TAKA-amylase and gluA promoters. Suitable promoters are mentioned in, e.g. EP 238 023 and EP 383 779.

The DNA sequence encoding the TFF monomer peptides may also, if necessary, be operably connected to a suitable terminator, such as the human growth hormone terminator (Palmiter et al., Science 222, 1983, pp. 809-814) or the TPI1 (Alber and Kawasaki, J. Mol. Appl. Gen. 1, 1982, pp. 419-434) or ADH3 (McKnight et al., The EMBO J. 4, 1985, pp. 2093-2099) terminators. The vector may further comprise elements such as polyadenylation signals (e.g. from SV40 or the adenovirus 5 Elb region), transcriptional enhancer sequences (e.g. the SV40 enhancer) and translational enhancer sequences (e.g. the ones encoding adenovirus VA RNAs).

The recombinant vector may further comprise a DNA sequence enabling the vector to replicate in the host cell in question. An example of such a sequence (when the host cell is a mammalian cell) is the SV40 origin of replication.

When the host cell is a yeast cell, suitable sequences enabling the vector to replicate are the yeast plasmid 2μ replication genes REP 1-3 and origin of replication.

The vector may also comprise a selectable marker, e.g. a gene the product of which complements a defect in the host cell, such as the gene coding for dihydrofolate reductase (DHFR) or the Schizosaccharomyces pombe TPI gene (described by P. R. Russell, Gene 40, 1985, pp. 125-130), or one which confers resistance to a drug, e.g. ampicillin, kanamycin, tetracyclin, chloramphenicol, neomycin, hygromycin or methotrexate. For filamentous fungi, selectable markers include amdS, pyrG, argB, niaD or sC.

To direct a TFF monomer peptide of the present invention into the secretory pathway of the host cells, a secretory signal sequence (also known as a leader sequence, prepro sequence or pre sequence) may be provided in the recombinant vector. The secretory signal sequence is joined to the DNA sequence encoding the TFF monomer peptide in the correct reading frame. Secretory signal sequences are commonly positioned 5′ to the DNA sequence encoding the peptide. The secretory signal sequence may be that, normally associated with the peptide or may be from a gene encoding another secreted protein.

For secretion from yeast cells, the secretory signal sequence may encode any signal peptide, which ensures efficient direction of the expressed TFF monomer peptide into the secretory pathway of the cell. The signal peptide may be naturally occurring signal peptide, or a functional part thereof, or it may be a synthetic peptide. Suitable signal peptides have been found to be the α-factor signal peptide (cf. U.S. Pat. No. 4,870,008), the signal peptide of mouse salivary amylase (cf. O. Hagenbuchle et al., Nature 289, 1981, pp. 643-646), a modified carboxypeptidase signal peptide (cf. L. A. Valls et al., Cell 48, 1987, pp. 887-897), the yeast BAR1 signal peptide (cf. WO 87/02670), or the yeast aspartic protease 3 (YAP3) signal peptide (cf. M. Egel-Mitani et al., Yeast 6, 1990, pp. 127-137).

For efficient secretion in yeast, a sequence encoding a leader peptide may also be inserted downstream of the signal sequence and upstream of the DNA sequence encoding the TFF monomer peptide. The function of the leader peptide is to allow the expressed peptide to be directed from the endoplasmic reticulum to the Golgi apparatus and further to a secretory vesicle for secretion into the culture medium (i.e. exportation of the TFF monomer peptide across the cell wall or at least through the cellular membrane into the periplasmic space of the yeast cell). The leader peptide may be the yeast α-factor leader (the use of which is described in e.g. U.S. Pat. No. 4,546,082, U.S. Pat. No. 4,870,008, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral α-amylase, A. niger acid-stable amylase, or A. niger glucoamylase. Suitable signal peptides are disclosed in, e.g. EP 238 023 and EP 215 594.

For use in insect cells, the signal peptide may conveniently be derived from an insect gene (cf. WO 90/05783), such as the lepidopteran Manduca sexta adipokinetic hormone precursor signal peptide (cf. U.S. Pat. No. 5,023,328).

The procedures used to ligate the DNA sequences coding for the TFF monomer peptide, the promoter and optionally the terminator and/or secretory signal sequence, respectively, and to insert them into suitable vectors containing the information necessary for replication, are well known to persons skilled in the art (cf., for instance, Sambrook et al., Molecular Cloning: A Laboratory Manual, Cold Spring Harbor, N. Y., 1989).

The host cell into which the DNA sequence encoding the TFF monomer peptide is introduced may be any cell, which is capable of producing the posttranslational modified TFF monomer peptide and includes yeast, fungi and higher eucaryotic cells.

Examples of suitable mammalian cell lines are the COS (ATCC CRL 1650), BHK (ATCC CRL 1632, ATCC CCL 10), CHL (ATCC CCL39) or CHO (ATCC CCL 61) cell lines. Methods of transfecting mammalian cells and expressing DNA sequences introduced in the cells are described in e.g. Kaufman and Sharp, J. Mol. Biol. 159 (1982), 601-621; Southern and Berg, J. Mol. Appl. Genet. 1 (1982), 327-341; Loyter et al., Proc. Natl. Acad. Sci. USA 79 (1982), 422-426; Wigler et al., Cell 14 (1978), 725; Corsaro and Pearson, Somatic Cell Genetics 7 (1981), 603, Graham and van der Eb, Virology 52 (1973), 456; and Neumann et al., EMBO J. 1 (1982), 841-845.

Examples of suitable yeasts cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous polypeptides there from are described, e.g. in U.S. Pat. No. 4,599,311, U.S. Pat. No. 4,931,373, U.S. Pat. Nos. 4,870,008, 5,037,743, and U.S. Pat. No. 4,845,075, all of which are hereby incorporated by reference. Transformed cells are selected by a phenotype determined by a selectable marker, commonly drug resistance or the ability to grow in the absence of a particular nutrient, e.g. leucine. A preferred vector for use in yeast is the POT1 vector disclosed in U.S. Pat. No. 4,931,373. The DNA sequence encoding the TFF monomer peptide may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (cf. Gleeson et al., J. Gen. Microbiol. 132, 1986, pp. 3459-3465; U.S. Pat. No. 4,882,279).

Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277, EP 238 023, EP 184 438 The transformation of F. oxysporum may, for instance, be carried out as described by Malardier et al., 1989, Gene 78: 147-156. The transformation of Trichoderma spp. may be performed for instance as described in EP 244 234.

When a filamentous fungus is used as the host cell, it may be transformed with the DNA construct of the invention, conveniently by integrating the DNA construct in the host chromosome to obtain a recombinant host cell. This integration is generally considered to be an advantage as the DNA sequence is more likely to be stably maintained in the cell. Integration of the DNA constructs into the host chromosome may be performed according to conventional methods, e.g. by homologous or heterologous recombination.

Transformation of insect cells and production of heterologous polypeptides therein may be performed as described in U.S. Pat. No. 4,745,051; U.S. Pat. No. 4,879,236; U.S. Pat. Nos. 5,155,037; 5,162,222; EP 397,485) all of which are incorporated herein by reference. The insect cell line used as the host may suitably be a Lepidoptera cell line, such as Spodoptera frugiperda cells or Trichoplusia ni cells (cf. U.S. Pat. No. 5,077,214). Culture conditions may suitably be as described in, for instance, WO 89/01029 or WO 89/01028, or any of the aforementioned references.

The transformed or transfected host cell described above is then cultured in a suitable nutrient medium under conditions permitting expression of the TFF monomer peptides after which all or part of the resulting peptide may be recovered from the culture. The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The TFF monomer peptides produced by the cells may then be recovered from the culture medium by conventional procedures including separating the host cells from the medium by centrifugation or filtration, precipitating the proteinaqueous components of the supernatant or filtrate by means of a salt, e.g. ammonium sulphate, purification by a variety of chromatographic procedures, e.g. ion exchange chromatography, gelfiltration chromatography, affinity chromatography, or the like, dependent on the type of polypeptide in question.

In the pharmaceutical composition of the invention, the TFF monomer peptides may be formulated by any of the established methods of formulating pharmaceutical compositions, e.g. as described in Remington's Pharmaceutical Sciences, 1985. The composition may be in a form suited for systemic injection or infusion and may, as such, be formulated with sterile water or an isotonic saline or glucose solution. The compositions may be sterilized by conventional sterilization techniques, which are well known in the art. The resulting aqueous solutions may be packaged for use or filtered under aseptic conditions and lyophilized, the lyophilized preparation being combined with the sterile aqueous solution prior to administration. The composition may contain pharmaceutically acceptable auxiliary substances as required to approximate physiological conditions, such as buffering agents, tonicity adjusting agents and the like, for instance sodium acetate, sodium lactate, sodium chloride, potassium chloride, calcium chloride, etc.

The pharmaceutical composition of the present invention may also be adapted for nasal, transdermal or rectal administration. The pharmaceutically acceptable carrier or diluent employed in the composition may be any conventional solid carrier. Examples of solid carriers are lactose, terra alba, sucrose, talc, gelatin, agar, pectin, acacia, magnesium stearate and stearic acid. Similarly, the carrier or diluent may include any sustained release material known in the art, such as glyceryl monostearate or glyceryl distearate, alone or mixed with a wax. The amount of solid carrier will vary widely but will usually be from about 25 mg to about 1 g.

The concentration of the TFF monomer peptides in the composition may vary widely, i.e. from from about 5% to about 100% by weight. A typical concentration is in the range of 50-100% by weight. A unit dosage of the composition may contain from about 1 mg to about 200 mg, typically from about 25 mg to about 75 mg, such as about 50 mg, of the peptide.

The term “a therapheutically effective amount” is the effective dose to be determined by a qualified practitioner, who may titrate dosages to achieve the desired response. Factors for consideration of dose will include potency, bioavailability, desired pharmacokinetic/pharmacodynamic profiles, condition of treatment (e.g. trauma, ulcerative colitis, gastric ulcers), patient-related factors (e.g. weight, health, age, etc.), presence of co-administered medications, time of administration, or other factors known to a medical practitioner. The dosage of a TFF monomer peptide administered to a patient will vary with the type and severity of the condition to be treated, but is generally in the range of 0.1-1.0 mg/kg body weight.

The term “subject” as used herein is intended to mean any animal, in particular mammals, such as humans, and may, where appropriate, be used interchangeably with the term “patient”.

### BRIEF DESCRIPTION OF THE DRAWINGS

The present invention is described in further detail in the examples with reference to the appended drawings wherein

FIG. 1. The mammalian Trefoil Factors (TFFs), TFF1 and TFF3 in monomer form. The figure shows the human sequences.

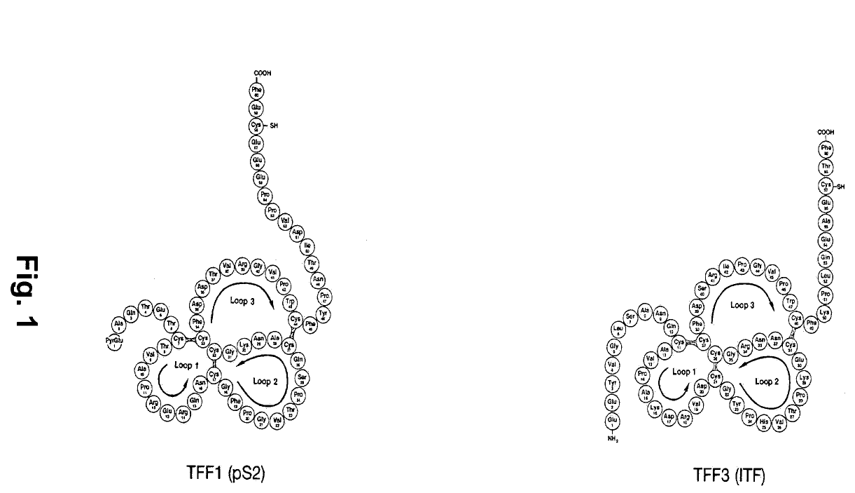

FIG. 2. Stress versus shear rate of mucin solution alone. 2 ml of 10% (w/v) mucin I dissolved in 0.05% (w/v) sodiumazide was added 0.4 ml of water. After 30 min at 200° C. the shear stress was measured as function of shear rate using the software programme: “constant rate—Approximation to power law.

FIG. 3. Viscosity versus shear rate of TFF3 monomer peptides. 2 ml 10% (w/v) mucin I dissolved in 0.05% sodiumazide was added 7.05 mg TFF3 monomer dissolved in water. After 30 min at 20° C. the viscosity was measured as function of shear rate using the software programme: “constant rate”. □—□: mucin I alone; ⋄—⋄: mucin I+TFF3 monomer; ●—●: TFF2.

The present invention is further illustrated by the following examples which, however, are not to be construed as limiting the scope of protection. The features disclosed in the foregoing description and in the following examples may, both separately and in any combination thereof, be material for realizing the invention in diverse forms thereof.

### EXAMPLES

### Example 1

Rheological Properties of TFF Peptides

Mucin I: Crude mucin, type II from porcine stomach (Sigma, St. Louis, Mo., USA). The monomer form of recombinant human TFF3 was prepared as previously described (Thim, L. et al.(1995) Biochemistry 34, 4757-4764). The monomer form of recombinant human TFF1 is prepared as previously described (Kannan, R. et al. (2001) Protein Expression and Purification 21, 92-98). Recombinant human Asn99-TFF2 in both glycosylated and the non-glycosylated forms were prepared in a yeast expression system as previously described (Thim, L. et al. (1993) FEBS Lett. 318, 345-352).

Mucin solutions. A 10% (w/v) solution of mucin I was prepared and TFF monomer peptides were dissolved in water and added to the mucin solution. After mixing the sample (Vortex mixer), the sample was allowed to stand for 5 min. and the viscosity was visually assessed in relation to a control solution of mucin added water without TFF monomer peptides. The detailed experimental conditions for the rheometer measurement are given in the figure legends.

Rheological measurements. Rheological properties were measured by the use of a rotational Reologica Rheometer (Reologica Instriments AB, Lund, Sweden). The instrument is equipped with a stainless steel C40 4 cone-plate (40 mm diameter plate with an angle of 4 degree) requiring a sample volume of at least 1.2 ml. The instrument was operated using instrument standard software (Version 3.6) allowing several different types of measurements. In the present study we have used the measuring programs: Constant Rate (viscosity and stress as a function of shear rate), Oscillation (complex viscosity, elastic modulus and viscous modulus at different frequencies) and Oscillation Stress Sweep (to identify the stress range inside which the measurement results are linear i.e. independent of the applied stress).

A visual assessment of the change in properties that could be observed when TFF monomer peptides was added to mucin solutions was made (Table 1). The addition of TFF monomer peptides to mucin solutions resulted in a significant visual decrease in viscosity when compared to the addition of TFF2 (Table 1).

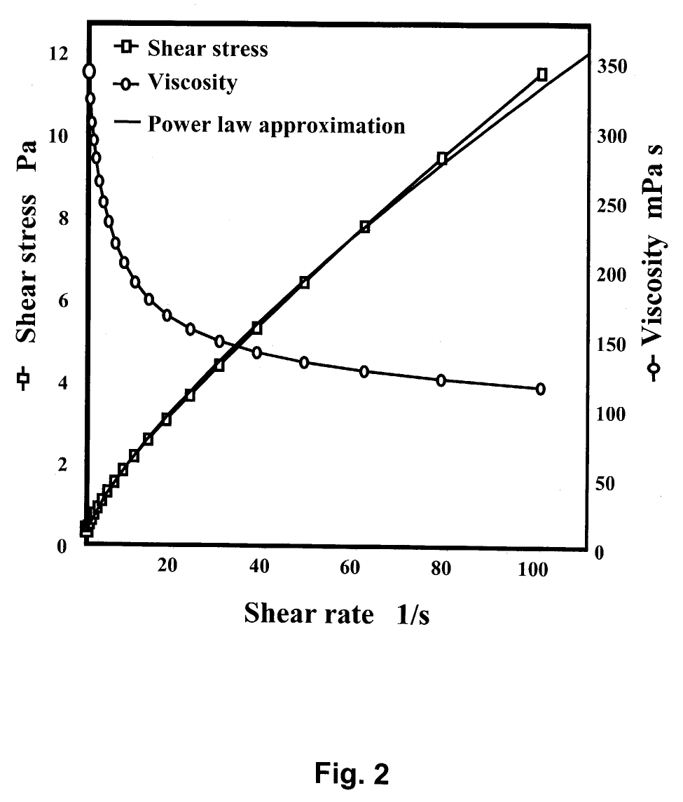

Mucin solutions and mucin/TFF monomer peptide gel-like substances. Mucin solutions to which a TFF monomer peptide was added was compared. As can be seen from FIG. 2 the mucin solution alone behaves as a non-Newtonian liquid. These liquids can be described by the Ostwald de Waele model (power law) (Barnes, H. A. (1989) An introduction to rheology. Elsevier and Ferguson, J. and Kemblowski, Z. (1991) Applied fluid rheology. Elsevier):

δ=k(γ)ⁿ,

where δ=shear stress, γ=shear rate and n and k are constants specific for the solution (if n=1 the solution is Newtonian). In the present case the following values could be calculated from FIG. 2: n=0.75 and k=0.35.

Since n<1 the solution is called shear-tinning, which is the characteristics of dispensions with asymmetric particles or emulsions. However, since the n value is close to 1 the solution is not far from being Newtonian. As can also be seen from FIG. 2 the viscosity varies from 0.34 Pa s at low shear rates to 0.12 Pa s at high shear rates.

In order to characterise the mucin/TFF monomer peptide gel-like structure, the technique of oscillatory measurement in which the gel-like material are subjected to a sinusoidally varying stress is applied and the strain response is measured. Before this measurement is carried out an oscillation stress sweep programme is used to define the so-called linear viscoelastic region. Inside this region no change of the mucin/TFF monomer peptide structure occurs and the relation between the applied stress and the measured quantities is linear. This type of experiments allow the estimation of several Theological parameters as a function of frequency: complex viscosity η*, elastic modulus G′ and viscous modulus G″ (for detailed rheological theory see Barnes, H. A. (1989) An introduction to rheology. Elsevier and Ferguson, J. and Kemblowski, Z. (1991) Applied fluid rheology. Elsevier)

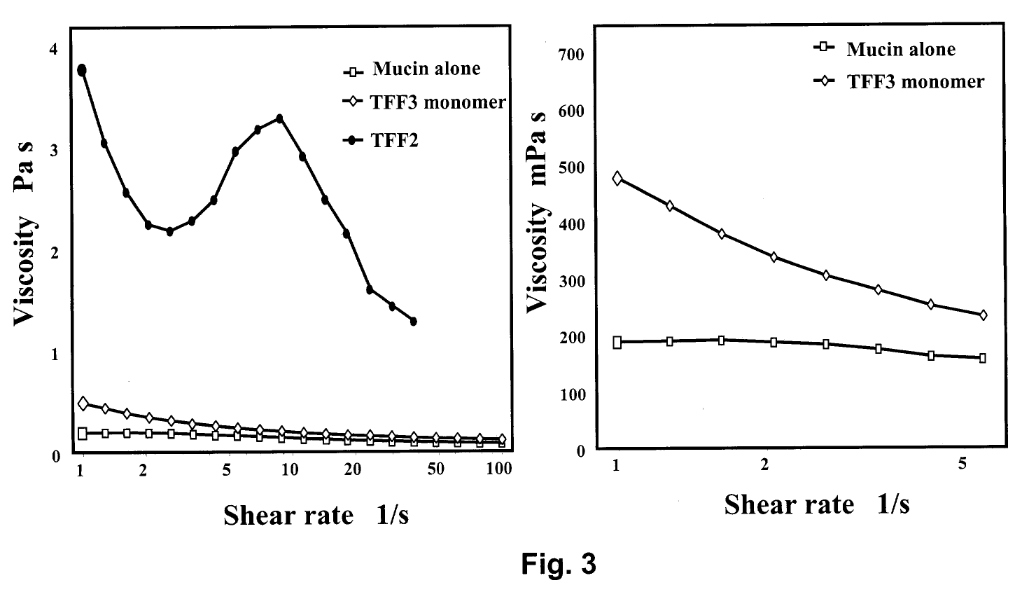

FIG. 3 shows the change in viscosity of the mucin I solution obtained by the addition of TFF3 monomer peptides. The TFF3 monomer peptides had a significant decreased effect on the viscosity of the mucin solution as compared to TFF2.

TABLE 1
Visual assessment of viscosity

| | | | Viscosity |
|---|---|---|---|
| Mucin I solution | TFF peptide | Amount TFF added | increase |
| 1 ml 10% (w/w) | TFF2 peptide | 7.5 mg in 200 μl | +++++ |
| 0.6 ml 10% (w/w) | TFF3 monomer | 5.8 mg in 100 μl | + |
| 0.6 ml 10% (w/w) | TFF3 monomer | 11.7 mg in 100 μl | + |

1. A pharmaceutical composition for decreasing the viscosity of mucus layers in mammals, the composition comprising a TFF monomer peptide or a pharmaceutically acceptable salt thereof.

2. The pharmaceutical composition according to claim 1, wherein the mammal is human.

3. The pharmaceutical composition according to any one of claims 1-2 for local and luminal application.

4. The pharmaceutical composition according to any one of claims 1-2 for parenteral administration.

5. The pharmaceutical composition according to any one of claims 1-2 for oral administration.

6. The pharmaceutical composition according to any one of claims 1-5, wherein the TFF monomer peptide is recombinant human TFF1.

7. The pharmaceutical composition according to any one of claims 1-5, wherein the TFF monomer peptide is recombinant human TFF3.

8. The pharmaceutical composition according to any one of claims 1-7, wherein the composition further comprises a mucin glycoprotein preparation.

9. The pharmaceutical composition according to any one of claims 1-8, for the treatment of oral mucosa.

10. The pharmaceutical composition according to claim 9, for the treatment of patients with reduced secretion of saliva.

11. The pharmaceutical composition according to claim 10, wherein the reduced secretion of saliva is caused by irradiation therapy, treatment with anticholinergics or Sjögrens syndrome.

12. The pharmaceutical composition according to any one of claims 1-8, for the treatment of the respiratory passages.

13. The pharmaceutical composition according to claim 12, for decreasing the viscosity of secretions in sinusitis or common cold causing nasal obstruction.

14. The pharmaceutical composition according to claim 12, for the treatment of the respiratory tract following accidental inhalation of irritants, gases, dusts or fumes.

15. The pharmaceutical composition according to claim 12, for the treatment of patients with allergic rhinitis.

16. The pharmaceutical composition according to claim 12, for the treatment of patients with diseases of the lungs causing viscous secretions and sputum, asthma, acute or chronic bronchitis, alpha-1 antitrypsin deficiency and cystic fibrosis.

17. The pharmaceutical composition according to any one of claims 1-8, for the treatment of the distal part of the oesophagus.

18. The pharmaceutical composition according to claim 17, for protection against acid secretions from the stomach in reflux oesophagi's, hiatus hernia or Barrets oesophagus.

19. The pharmaceutical composition according to any one of claims 1-8, for the treatment of the stomach.

20. The pharmaceutical composition according to claim 19, for treatment of stress induced gastric ulcers secondary to trauma, shock, large operations, renal or lever diseases, or treatment with aspirin, other NSAIDS, steroids or alcohol.

21. The pharmaceutical composition according to any one of claims 1-8, for the treatment of obstipation.

22. The pharmaceutical composition according to any one of claims 1-8, for the treatment of the small intestinal, large intestine or colonic mucosa in Crohns disease, ulcerative colitis, pseudomembranous colitis, irritable bowel syndrome, and cystic fibrosis.

23. The pharmaceutical composition according to any one of claims 1-8, for the treatment of the eye.

24. The pharmaceutical composition according to claim 23, for decreasing the viscosity of lacrimal fluid in patients with keratoconjunctivitis sicca/Sjögren's syndrome or dry eyes.

25. The pharmaceutical composition according to any one of claims 23-24, wherein the pharmaceutical composition is in eye droplets.

26. The pharmaceutical composition according to any one of claims 1-8, for the treatment of the joints.

27. The pharmaceutical composition according to claim 26, for decreasing the viscosity of the synovial fluid in osteoarthritis and following joint replacement.

28. The pharmaceutical composition according to any one of claims 1-8, for the treatment of chronic bladder infections, patients with catheter, interstitial cystitis, papillomas or cancer of the bladder.

29. The pharmaceutical composition according to any one of claims 1-8, for the treatment of the urogenital system.

30. The pharmaceutical composition according to any one of claims 1-8, for the treatment of the uterine cervix.

31. The pharmaceutical composition according to any one of claims 1-8, for the treatment infertility.

32. Use of a TFF monomer peptide for the preparation of a medicament for decreasing the viscosity of mucus layers in mammals.

33. Use of a TFF monomer peptide for the preparation of a medicament for decreasing the viscosity of mucus layers in mammals, wherein the medicament is according to any one of the claims 1-31.

34. Use according to any one of the claims 32-33, wherein the mammal is human.

35. A method for in vivo decrease in viscosity of mucus layers in a subject, said method comprising administering to the subject a composition comprising
a) a pharmaceutically acceptable carrier or diluent,
b) a therapheutically effective amount of a TFF monomer peptide, and optionally
c) a mucin glycoprotein preparation.

36. The method according to claim 35, wherein the administration is local and luminal.

37. The method according to claim 35, wherein the administration is parenteral.

38. The method according to any one of the claims 35-37, wherein the TFF monomer peptide is recombinant human TFF1.

39. The method according to any one of the claims 35-37, wherein the TFF monomer peptide is recombinant human TFF3.

40. The method according to any one of the claims 35-39, wherein the mucin viscosity levels are associated with a disease state in the oral mucosa.

41. The method according to claim 40, wherein the disease state is a reduced secretion of saliva.

42. The method according to claim 41, wherein the reduced secretion of saliva is caused by irradiation therapy, treatment with anticholinergics or Sjögrens syndrome.

43. The method according to any one of the claims 35-39, wherein the mucin viscosity levels are associated with a disease state in the respiratory passages.

44. The method according to claim 43, wherein the disease state is sinusitis or common cold causing nasal obstruction.

45. The method according to claim 43, wherein the disease state is accidental inhalation of irritants, gases, dusts or fumes.

46. The method according to claim 43, wherein the disease state is allergic rhinitis.

47. The method according to claim 43, wherein the disease state is diseases of the lungs causing viscous secretions and sputum, asthma, acute or chronic bronchitis, alpha-1 antitrypsin deficiency and cystic fibrosis.

48. The method according to any one of the claims 35-39, wherein the mucin viscosity levels are associated with a disease state in the distal part of the oesophagus.

49. The method according to claim 48, wherein the disease state is acid secretions from the stomach in reflux oesophagi's, hiatus hernia or Barrets oesophagus.

50. The method according to any one of the claims 35-39, wherein the mucin viscosity levels are associated with a disease state in the stomach.

51. The method according to claim 50, wherein the disease state is stress induced gastric ulcers secondary to trauma, shock, large operations, renal or lever diseases, or treatment with aspirin, other NSAIDS, steroids or alcohol.

52. The method according to any one of the claims 35-39, wherein the disease state is obstipation.

53. The method according to any one of the claims 35-39, wherein the mucin viscosity levels are associated with a disease state in the small intestine, large intestine or colon.

54. The method according to claim 53, wherein the disease state is Crohns disease, ulcerative colitis, pseudomembranous colitis, irritable bowel syndrome, and cystic fibrosis.

55. The method according to any one of the claims 35-39, wherein the mucin viscosity levels are associated with a disease state in the eye.

56. The method according to claim 55, wherein the disease state is keratoconjunctivitis sicca/Sjögren's syndrome or dry eyes.

57. The method according to any one of the claims 35-39, wherein the mucin viscosity levels are associated with a disease state in the joints.

58. The method according to claim 57, wherein the disease state is increased viscosity of the synovial fluid in osteoarthritis or following joint replacement.

59. The method according to any one of the claims 35-39, wherein the disease state is chronic bladder infections, patients with catheter, interstitial cystitis, papillomas or cancer of the bladder.

60. The method according to any one of the claims 35-39, wherein the disease state is in the urogenital system.

61. The method according to claim 60, wherein the disease state is in the uterine cervix.

62. The method according to claim 60, wherein the disease state is infertility.
